(19) United States
(12) Patent Application Publication
(10) Pub. No.: US 2010/0130843 A1
(43) Pub. Date: May 27, 2010

(54) WIRELESS DEVICE FOR CONFIRMATORY DIAGNOSIS OF CYSTIC FIBROSIS THROUGH ANALYSIS OF SWEAT CHLORIDE

(75) Inventors: Alejandro Caceres Galvez, Sant Celoni (Barcelona) (ES); Rosa Passarell Serred, Sant Celoni (Barcelona) (ES)

Correspondence Address: EGBERT LAW OFFICES, 412 MAIN STREET, 7TH FLOOR, HOUSTON, TX, 77002, US

(73) Assignee: TECNICAS CIENTIFICAS PARA LABORATORIO, S.A, Barcelona (ES)

(21) Appl. No.: 12/277,273

(22) Filed: Nov. 24, 2008

(51) Int. Cl. A61B 5/145

(52) U.S. Cl. 600/346

(57) ABSTRACT

Wireless equipment for confirmatory diagnosis of cystic fibrosis through sweat testing, includes a module of analyzer/transmitter, with a box, built-in microprocessor for sweat induction analysis through chloride measurement device, radio antenna with a transmitter, battery, switch, and sensor card wiring connector with a single-use. The card has two iontophoresis electrodes, and ion-selective electrodes, built-in connector to the module, and a module terminal/receiver. The box (4) has a desktop keyboard, a LCD, microprocessor, USB connections for peripherals, radio antenna with receiver to get data “live” from the analyzer module/transmitter. The device is powered by batteries or wall electrical socket.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The invention describes a wireless device for confirmatory diagnosis of cystic fibrosis, through sweat analysis, of pediatric patients. A number of innovative and advantageous features are provided by the present invention, which will be described in detail. Together they pose a significant improvement over what was already known in this field.

2. Description of Related Art Including Information Disclosed Under 37 CFR 1.97 and 37 CFR 1.98

Cystic fibrosis is an autosomal and multisystem disease, which among other symptoms, is characterized by altered concentration of electrolytes in secretion.

As with many other diseases, their early detection is a key point for treatment, because it allows a clinical control of it, being their diagnosis confirmed by running sweat test.

In summary form, the sweat test consists of three main phases:
Producing the sweat sample. The preferred technique is recognized as iontophoresis with pilocarpine. A cholinergic drug coupled to the pilocarpine stimulates sweat glands. Sweat obtained in this way is functionally identical to sweat produced physiologically.
Collecting the sweat sample is a very sensitive procedure. Because only a small amount of sweat is collected, it must be done without suffering any contamination and without any evaporation.
Analysis of sweat sample. There are three groups of methods currently employed: quantitative methods require accurate measurement of sample weight, elution of the sample from absorbent gauze, and a time-consuming manual titration specific for chloride ions. This is the classic Gibson Cooke method, which is no longer used. Ion selective electrodes have been tried, but no available equipment can perform the analysis on microliter quantities of the sample. Semi-quantitative, and non-specific methods, determine the electrolyte concentration by conductivity, and purely qualitative methods are available, based on visual analysis of color change.

For more than ten years, the field of sweat analysis has been without any kind of innovative technological advancement, rendering much of the available equipment and instrumentation obsolete.

The general problem of existing methodologies is that they are very laborious; pose a nuisance for the patient, in this case children, and lack needed sensitivity for a conclusive result.

On the other hand, all the existing equipment for analysis operated with cables connected to the patient, except those that are wireless at the stage of obtaining sweat. The system used to perform the analysis is not confirmatory, which means that instruments are inaccurate for determining the level of chloride in the analysis of sweat.

It should be noted, therefore, that the existence of a wireless device for measuring chloride in sweat, applied to the detection of cystic fibrosis is unknown to the inventor. A technical and structural constituency is submitted similar to those advocated by the present invention, which aims to provide answers to the problems, described above, using a method based on an ion selective electrode, which detects the ions for which they were designed. The invention also greatly decreases the problems arising from the sample's manipulation that may occur during the collection of sweat.

Specifically, the object of the invention consists of a wireless device to measure concentration of chloride in the sweat of patients with symptoms, family history, or genetic profile suggestive of cystic fibrosis. Analysis is by through ion selective electrode, using the principle of iontophoresis with pilocarpine to obtain the sweat. Unlike methods currently employed for sweat analysis, the present invention does not actually collect the sweat. It performs the measurement at the point of emission through a selective electrode, placed in the area of stimulation, and which also determines in situ the quantitative value of chloride concentration.

### BRIEF SUMMARY OF THE INVENTION

Thus, the equipment proposed by the invention is by itself an obvious novelty within its scope. Accordingly, the present invention is a portable and self-contained instrument for diagnosis and confirmation of cystic fibrosis through the simultaneous use of the chloride ion selective sensors and impedance microsensors. The system is implemented for stimulating the production of sweat in a faster way and at lower cost. This sensitive system of detection is based on a disposable card, meaning that the invention can be a wireless apparatus for performing the sweat test.

The equipment is comprised of two individual units.

First, there is a remote unit, which integrates the sweat generator. The device performs iontopheresis of pilocarpine, the chloride measuring electrodes, and a radio transmitter for sending the analysis information.

Second, there is a receiver unit for collecting data from the remote analyzer module, displaying and recording test results in the internal data base memory.

The remote unit consists of a box or case, fitted with Velcro (TM) straps suitable for coupling to the body area of the patient. Electronics are incorporated inside, having a microprocessor that is responsible for doing all the tasks described above, a radio antenna for remote-unit communication, and internal wiring that connects this module with the single-use sensor card that will be placed at its base.

This disposable sensor card, at the same time, consists of two electrodes that allow the generation of sweat by specific reagent incorporated and using the technique called iontophoresis, two electrodes that allow the analysis of the sweat generated by the iontophoresis system, capable of determining the concentration of chloride ions, and a connection port that allows the card to be connected to the module to form a single piece.

With regard to the receiver unit, the receiver unit is composed of a desktop box or case, which houses all electronic components and provides a screen of liquid crystal display (LCD). All data is provided from the remote unit, including status indication, fault analysis, communication with peripheral devices, such as a printer and/or a computer connected to it via USB port, and a keyboard that, among other functions, allows the user to move through the menu generated by software, for example, to change setup values, such as starting points of operation, merging functions, and selecting conversion factors, in addition to assuring the proper functioning of peripheral devices.

The receiver unit also has a radio antenna to communicate in bidirectional mode with remote unit.

This wireless equipment confirms the diagnosis of cystic fibrosis through sweat testing suggested by the invention. A selective ion electrode allows the user to advantageously detect the concentration of an analyte of sweat in concentrations as low as 5 mmol/l, because this electrode is designed to be highly selective, discriminating against other ions that are typically found in sweat.

This fact makes the equipment of the invention much more reliable when it has to give a reading, compared with currently used equipment based only in conductivity method, which can have more false readings due to interference in the analyte, because this system only measures the ease with which a sample of sweat allows the transport of electricity through it. Therefore, by increasing the level of salts in the sample, the conductivity increases significantly, and although all the ionic salts are good conductors of electricity, the analyte that is required to be verified, in order to see whether a child has cystic fibrosis or not, is only found in a certain type of salt.

The new wireless equipment for confirmatory diagnosis of cystic fibrosis testing through sweat analysis, is therefore an innovative invention with structural characteristics and constituents unknown so far. Together with its practical usefulness, the present invention has sufficient basis for the privilege of exclusivity that is sought.

### BRIEF DESCRIPTION OF THE SEVERAL VIEWS OF THE DRAWINGS

To complement the description being used and in order to provide better understanding of the characteristics of the invention, a set of drawings is accompanying this description, as an integral part thereof. The set of drawings are illustrative and not limiting.

FIG. 1 shows a schematic view of the wireless equipment for confirmatory diagnosis of cystic fibrosis testing, through sweat analysis, according to the invention, which illustrate the main elements thereof.

FIG. 2 shows a schematic view of the bottom of the remote unit, showing the disposable sensor card as well as the elements integrated with the card.

### DETAILED DESCRIPTION OF THE INVENTION

In view of the above figures and reference numerals herein, the drawings illustrate an example of a preferred embodiment of the wireless equipment for confirmatory diagnosis of cystic fibrosis testing through sweat analysis, which includes the parts that are listed and described in detail below.

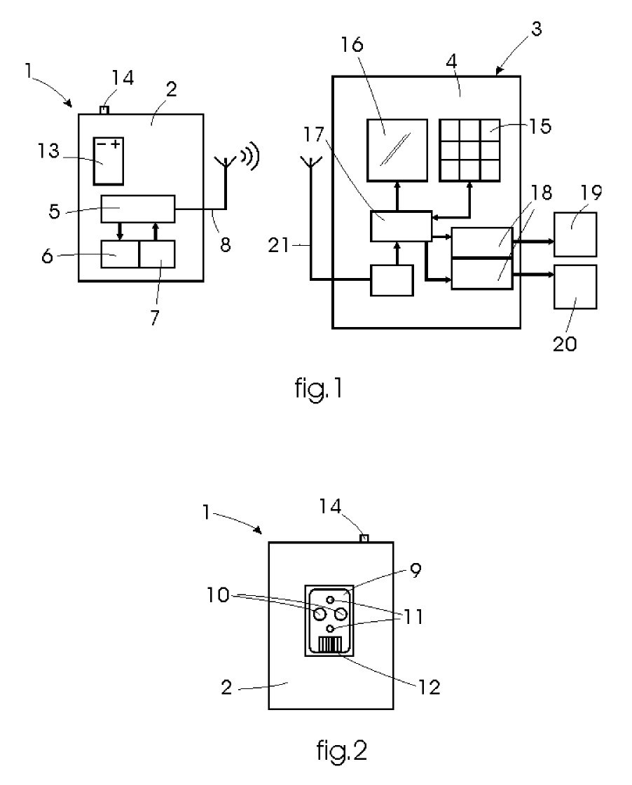

Thus, as shown in FIG. 1, the equipment consists of a remote unit (1), inside a box or case (2), fitted with Velcro™ for coupling to the body area of the patient's convenience, and a receiver unit (3), based on a benchtop box or case (4) that hosts all of its internal components and is intended to receive the data coming from remote unit (1), display and non-volatile memory, both remote and base modules are connected by radio waves.

The box (2) remote unit (1) includes an analog microprocessor (5), which is responsible for carrying out the functions of generation of sweat (6) and analysis by measuring chloride (7), a radio antenna (8) with its corresponding remote unit, and internal wiring that enables connection to an sensor card (9) single-use sensor, located at its base. As shown in FIG. 2, two iontophoresis electrodes (10) are provided, leading pilocarpine nitrate, which penetrates the skin and stimulates sweat. Two ion selective electrodes (11) enable the analysis of the sweat generated by the above iontophoresis electrodes (10), and an area of connection (12) that allows the sensor card to connect to the module.

It should be noted that the remote analyzer module/transmitter (1) is battery powered (13) and contains as well an intelligent switch (14) for the choice of several options.

For its part, the box (4) terminal/receiver (3) incorporates a keyboard (15), allowing navigation in the menu generated by the software tool, LCD screen (16) where all the data processed is displayed from analyzer module/transmitter (1), a microprocessor (17), equipped with software that controls all functions, as well as one or more USB connections (18) for peripheral devices such as a printer (19) and/or a computer (20).

The module terminal/receiver (3) also has a radio antenna (21) with its corresponding receiver module to communicate data bidirectional from the analyzer module/transmitter (1). It can be powered by a conventional battery (not shown) or plugged into a wall socket.

Sufficiently described the nature of this invention, as well as how to implement it, it should be noted that, within its essence, can be implemented in other forms of conduct that could be different than this one used as an example, and for which the protection is sought, provided that no alteration, change or modification of its fundamental principle.

It could be possible to extend the system to measure others ions or substances excreted by sweat, that can be analyzed directly on the skin by direct potentiometry or any other method of analysis.

1. Wireless equipment for confirmatory diagnosis of cystic fibrosis through sweat testing, measuring chloride in sweat concentration by a system of selective ions, using the principle of iontophoresis with pilocarpine, for sweat induction, the equipment comprising:
a module analyzer/transmitter, being fitted in a case and being provided with positioning straps;
a sweat generator, being attached to the module analyzer;
a sweat generated analyzer and data transmitter, having a selective electrode performing measurement when directly placed on a stimulation area; and
a module terminal/receiver compound being fitted into a compound case and having components therein for data reception coming from the analyzer module/transmitter, display and record in memory, the module analyzer and the module compound being connected by radio frequency.

2. Wireless equipment for confirmatory diagnosis of cystic fibrosis testing based on sweat, according to claim 1, further comprising:
a microprocessor to handle and perform sweat generation functions being housed in said case for the module analyzer;
a sweat testing sensor device for chloride measurement; and
a radio antenna with a corresponding quantitative ion chloride concentration transmitter in communication with the module; compound and being powered by battery and regular mains voltage socket and a switch built with wiring allowing connection to an analyzer card of a single use.

3. Wireless equipment for confirmatory diagnosis of cystic fibrosis testing in sweat, according to claim 2, wherein said analyzer card comprises two iontophoresis electrodes with pilocarpine nitrate, one or more selective ion electrodes, and a connection area to the module analyzer.

4. Wireless equipment for confirmatory diagnosis of cystic fibrosis testing through sweat, according to claim 1, further comprising:
components housed in said compound case, said components being comprised of a keyboard to choose menu selections generated by the software, an LCD screen where all data from the analyzer module/transmitter are displayed, a microprocessor, equipped with software that controls all functions, as well as one or more USB connections, for connecting peripherals, said microprocessor having a radio antenna with a corresponding receiver module sending commands to, and receiving data from analyzer module/transmitter, also powered by batteries or by connecting to regular wall electrical socket.
